(19) United States
(12) Patent Application Publication
(10) Pub. No.: US 2025/0248897 A1
(43) Pub. Date: Aug. 7, 2025

(54) CONTAINER WITH INTERNAL PORT

(71) Applicant: Instant Systems, Inc., Norfolk, VA (US)

(72) Inventors: Uzair Ahmed RAJPUT, Virginia Beach, VA (US); Emilio Alejandro EDWARDS, Norfolk, VA (US)

(73) Assignee: Instant Systems, Inc., Norfolk, VA (US)

(21) Appl. No.: 18/855,853

(22) PCT Filed: Apr. 13, 2023
(86) PCT No.: PCT/US2023/018535

§ 371 Date: Oct. 10, 2024

Related U.S. Application Data
(60) Provisional application No. 63/332,231, filed on Apr. 18, 2022.

(51) Int. Cl. A61J 1/14; A61J 1/10; C12M 1/00; C12M 1/12

(52) U.S. Cl. CPC A61J 1/1475; A61J 1/10; C12M 23/26; C12M 23/34; C12M 25/04

(57) ABSTRACT

A flexible container for storing a biological material includes a first layer coupled to a second layer via a set of seals to define a storage volume. The first layer and the second layer defining an opening therebetween. The opening extending into the storage volume. A flexible container also including an internal port that extends from the opening into the storage volume.

### CROSS-REFERENCE TO RELATED APPLICATIONS

This patent application claims priority to and the filing date benefit of U.S. Provisional Application Ser. No. 63/332,231, filed Apr. 18, 2022, entitled “Container with Internal Port,” which is incorporated herein by reference in its entirety.

### BACKGROUND

The embodiments described herein relate to containers for storing, processing, manipulating, packaging, and transporting biological material (including cells, cell culture media, food products, tissue) and processing solutions used in connection with biologic material.

In cellular material storage applications, layers of the bags used to store cells frequently stick together. Flexible containers for storing, processing, and manipulating cells are constructed from two layers of polymeric material. After production, the properties of the material and the lack of air within the bag (e.g., resulting from sterilization, manufacture) can cause the two layers to stick together. This causes the internal volume within which the cells are to be contained to not fully expand or otherwise be unavailable to receive the cells. There are several known methods for reducing sticking of opposing layers and expanding the storage volume. One method is by increasing the pressure of the fluid being conveyed via a port into the container. This can damage cells due to increased pressure on cell walls and increased shear stress on the cells. Increasing the pressure can also result in high and uneven flow rates of fluid into the bag. Because many cell storage, processing, and manipulating procedures require accurate quantities, increasing the pressure (and flow rate) can reduce accuracy of the volume of cells delivered. Another known method for reducing sticking can include providing an additive material (e.g., a powder or other chemical) between the layers to reduce the adherence of the layers. Adding materials to an otherwise inert bag that defines a sterile environment, however, can be damaging to cells. Another known method of preventing polymeric layers from sticking together is adding a removable lint-free paper or liner between the layers. Because containers for storing cells are closed systems (with one or more ports, but without a long, open edge), there would be no reasonable way to pull out the liner prior to use.

Traditionally, cell storage bags are produced from polymeric materials that are gas permeable to facilitate gas exchange for cell growth and to maintain viability. The gas exchange is controlled by a number of factors including the thickness of the layers and the surface area of the layers. Some known containers do not provide sufficient gas exchange. It can be desirable to improve the gas exchange for a given internal volume/surface area ratio.

Materials stored in flexible containers are frozen and thawed for storage and processing. It is desirable that the process for freezing and thawing be done in a controlled, uniform manner. Heat is generally transferred into (or out of) a container only via the outer layers. This can produce some spatial nonuniformity of temperature within the material (with the interior portions being slower to thaw, etc.).

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In one aspect, the present disclosure is directed to a flexible container for storing a biological material. In some embodiments, the flexible container includes a first layer coupled to a second layer via a set of seals to define a storage volume. The set of seals defines an opening between the storage volume and an external environment. The flexible container also includes an internal port positioned within the storage volume and in fluid communication with the opening. The internal port is positioned to maintain a separation volume between the first layer and the second layer. The internal port has a wall that defines an interior passage. The interior passage is in fluid communication with the storage volume.

In some embodiments, the internal port facilitates a gas transfer into the storage volume.

In some embodiments, the interior passage is in fluid communication with the storage volume via a gas-permeable portion of the wall of the internal port.

In some embodiments, at least one of the first layer or the second layer is gas permeable. However, in some embodiments the first layer and the second layer are gas impermeable.

In some embodiments, the internal port is fluidly coupled to an aeration filter.

In some embodiments, the wall of the internal port defines an aperture between the interior passage and the storage volume.

In some embodiments, the internal port is fluidly coupled to a connector unit, and the connector unit is configured to be fluidly coupled to a gas source and to a biological material source.

In some embodiments, the connector unit includes a flow control member. In a first state, the flow control member is configured to place the storage volume in fluid communication with the biological material source to facilitate delivery of the biological material from the biological material source and into the storage volume. In a second state, the flow control member is configured to place the storage volume in fluid communication with the gas source to facilitate the gas transfer.

In some embodiments, the flexible container includes a sleeve positionable within the interior passage and configured to occlude the aperture, the sleeve being gas permeable and impermeable to the biological material.

In some embodiments, the flexible container includes a sleeve positionable within the interior passage, the sleeve being configured to transition the internal port from a first transmission state to a second transmission state. In the first transmission state, the internal port is configured to deliver the biological material into the storage volume. In the second transmission state, the internal port is configured to convey a treatment media. In some embodiments, the sleeve is gas permeable and impermeable to the biological material.

In some embodiments, the internal port is a first internal port and the opening is a first opening and the flexible container includes a second internal port positioned within the storage volume and in fluid communication with a second opening defined by the set of seals, the second internal port having a wall that defines an interior passage that is in fluid communication with the storage volume.

In some embodiments, the first internal port is configured to facilitate a first procedure, and the second internal port is configured to facilitate a second procedure. The first procedure is different from the second procedure.

In some embodiments, the first procedure corresponds to a delivery of the biological material into the storage volume, and the second procedure corresponds to a removal of the biological material from the storage volume.

In some embodiments, the first procedure corresponds to a delivery of the biological material into the storage volume and a removal of the biological material from the storage volume; and the second procedure corresponds to a conveying of a treatment media.

In some embodiments, the internal port extends from the opening into the storage volume.

In some embodiments, the internal port extends through a seal of the set of seals defining the opening.

In some embodiments, the flexible container includes an external fitting coupled to the internal port, the external fitting being outside the storage volume.

In some embodiments, the internal port and the external fitting form a contiguous component, and the contiguous component is coupled to the first layer and the second layer at the opening.

In some embodiments, the internal port is configured to facilitate conveyance of a treatment media.

In some embodiments, the treatment media is a heat transfer media, and the heat transfer media is configured to affect a temperature within the storage volume.

In some embodiments, the wall of the internal port includes a conductive material configured to transfer energy between the heat transfer media and the biological material within the storage volume.

In some embodiments, at least one of the first layer or the second layer is gas permeable, and the gas permeability facilitates a gas transfer between the storage volume and an external environment.

In some embodiments, the internal port facilitates at least one of a delivery of the biological material into the storage volume or a removal of the biological material from the storage volume. The interior passage has a cross-sectional area sized to facilitate passage of the biological material. The wall of the internal port defines an aperture between the interior passage and the storage volume, and the aperture is sized to facilitate passage of the biological material therethrough.

In some embodiments, the aperture is a single aperture defined by an end of the internal port within the storage volume.

In some embodiments, the aperture is an aperture of a set of apertures defined by the wall of the internal port.

In some embodiments, the set of apertures includes a set of aperture pairs. Each aperture pair includes two opposing apertures that are axially aligned at a single longitudinal position. The set of aperture pairs is distributed longitudinally along the internal port.

In some embodiments, each aperture of the set of apertures is arranged as a single aperture. Each aperture is axially misaligned with the remaining apertures of the set of apertures. The set of apertures is distributed longitudinally along the internal port and circumferentially about the internal port.

In some embodiments, the set of apertures includes apertures of different diameters.

In some embodiments, the apertures of the set of apertures positioned at a first distance relative to the opening have a first diameter, while the apertures of the set of apertures positioned at a second distance relative to the opening have a second diameter that is greater than the first diameter. The second distance is greater than the first distance.

In some embodiments, the apertures of the set of apertures positioned at a first distance relative to the opening have a first diameter, while the apertures of the set of apertures positioned at a second distance relative to the opening have a second diameter that is less than the first diameter. The second distance is greater than the first distance.

In some embodiments, the wall of the internal port defines an uninterrupted portion adjacent to the opening, and the aperture is positioned between the uninterrupted portion and an end of the internal port within the storage volume.

In some embodiments, the aperture is positioned in contact with the seal.

In some embodiments, the internal port has a greater rigidity than a rigidity of the first layer and a rigidity of the second layer.

In some embodiments, the internal port has a fixed cross-sectional shape.

In some embodiments, the internal port has a first cross-sectional shape on a condition that the storage volume does not contain the biological material. The internal port is configured to have a second cross-sectional shape on a condition that the storage volume contains the biological material. The first cross-sectional shape is different from the second cross-sectional shape.

In some embodiments, the storage volume has a longitudinal length, and the internal port has a length that is greater than 25 percent of the longitudinal length of the storage volume and less than 95 percent of the longitudinal length of the storage volume.

In some embodiments, the opening is a first opening in fluid communication with the internal port, and the flexible container includes an external port. The external port defines a second opening in the set of seals.

In some embodiments, the external port and the second opening are positioned at a first end portion of the flexible container; and the first opening is positioned at a second end portion of the flexible container.

In some embodiments, the first opening and the second opening are axially aligned.

In some embodiments, the internal port facilitates delivery of the biological material into the storage volume, and the external port facilitates removal of the biological material from the storage volume.

In some embodiments, the second layer is one of a rigid material or a rigid laminate, and the second layer is a support structure configured to support the biological material in the storage volume.

In some embodiments, the opening in fluid communication with the internal port is positioned at a second end portion of the flexible container. The flexible container includes a sealable opening defined by the first layer and the second layer. The sealable opening extends between a first side of the flexible container and a second side of the flexible container. The sealable opening is positioned at a first end portion of the flexible container. The sealable opening is configured to facilitate an introduction of the biological material into the storage volume and a removal of the biological material from the storage volume. Further, the sealable opening is configured to form a seal following the introduction of the biological material into the storage volume.

In some embodiments, the seal is a peelable seal that hermitically seals the storage volume. The peelable seal is configured such that the first layer can be peeled away from the second layer to expose the storage volume.

In some embodiments, at least one of the internal port, the first layer, or the second layer include at least one marking, the marking being configured to indicate a volume of the biological material within the storage volume.

In some embodiments, the storage volume is a first storage volume, and the flexible container includes a second storage volume defined by the first layer and the second layer, the second storage volume being separated from the first storage volume by a seal. In some embodiments, the seal separating the first storage volume from the second storage volume is a permeable seal.

In some embodiments, the internal port is a first internal port within the first storage volume and the opening is a first opening. The flexible container further includes a second internal port positioned within the second storage volume and in fluid communication with a second opening defined by the set of seals. The second internal port has a wall that defines an interior passage that is in fluid communication with the second storage volume.

In some embodiments, the opening is a first opening. The flexible container includes a second opening defined by the set of seals. The internal port extends between a first end portion and a second end portion. The first end portion is in fluid communication with the first opening. The second end portion is in fluid communication with the second opening. In some embodiments, the first opening and the second opening are at a first end portion of the flexible container.

In some embodiments, the internal port has a serpentine configuration within the storage volume, and the internal port has a length that is greater than a maximal longitudinal length of the storage volume.

In some embodiments, the flexible container includes a removable plug. The removable plug is configured to temporarily occlude one of the first end portion or the second end portion of the internal port.

In some embodiments, the internal port is at least partially formed from a shape memory material. The internal port has a first shape at a first temperature and a second shape at a second temperature that is different from the first temperature, with the first shape being different from the second shape.

In another aspect, the present disclosure is directed to a flexible container for storing a biological material. In some embodiments, the flexible container includes a first layer coupled to a second layer via a set of seals to define a storage volume. The set of seals defines a first opening and a second opening between the storage volume and an external environment. Additionally, the flexible container includes an internal port positioned within the storage volume. The internal port has a first end portion in fluid communication with the first opening and a second end portion in fluid communication with the second opening. The internal port is positioned to maintain a separation volume between the first layer and the second layer. The internal port has a wall that defines an interior passage in fluid communication with the first opening and the second opening.

In some embodiments, the internal port facilitates a gas transfer into the storage volume.

In some embodiments, the interior passage is in fluid communication with the storage volume via a gas-permeable portion of the wall of the internal port.

In some embodiments, at least one of the first layer or the second layer is gas permeable. However, in some embodiments, the first layer and the second layer are gas impermeable.

In some embodiments, the interior passage is configured to receive a treatment media; and the internal port is positioned to convey the treatment media through the storage volume to affect the biological material in the storage volume.

In some embodiments, the interior passage of the internal port is fluidically isolated from the storage volume, and the treatment media is a heat transfer media. The heat transfer media is configured to affect a temperature within the storage volume.

In some embodiments, the wall of the internal port includes a conductive material configured to transfer energy between the heat transfer media and the biological material with the storage volume.

In some embodiments, the first opening and the second opening are at a first end portion of the flexible container.

In some embodiments, the flexible container includes an external port. The external port defines a third opening in the set of seals. The external port is configured to facilitate an introduction of the biological material into the storage volume and a removal of the biological material from the storage volume. In some embodiments, the external port and the third opening are positioned at a first end portion of the flexible container, and the first opening and the second opening are positioned at a second end portion of the flexible container.

In some embodiments, at least one of the first opening or the second opening is positioned at a second end portion of the flexible container. The flexible container further includes a sealable opening defined by the first layer and the second layer. The sealable opening extends between a first side of the flexible container and a second side of the flexible container, the sealable opening is positioned at a first end portion of the flexible container, is configured to facilitate an introduction of the biological material into the storage volume and a removal of the biological material from the storage volume, and is configured to form a seal following the introduction of the biological material into the storage volume. In some embodiments, the seal is a peelable seal that hermitically seals the storage volume, and is configured such that the first layer can be peeled away from the second layer to expose the storage volume.

In some embodiments, the second layer is one of a rigid material or a rigid laminate, and the second layer is a support structure configured to support the biological material in the storage volume.

### BRIEF DESCRIPTION OF THE DRAWINGS

FIG. 1A is a schematic illustration of a storage container according to an embodiment.

FIG. 1B is a schematic illustration of a storage container according to an embodiment.

FIG. 1C is a cross-sectional view of the storage container of FIG. 1A taken at line x-x in FIG. 1A and depicting the internal port in a first configuration according to an embodiment.

FIG. 1D is a cross-sectional view of the storage container of FIG. 1A taken at line x-x in FIG. 1A and depicting the internal port in a second configuration according to an embodiment.

FIG. 1E is a cross-sectional view of the storage container of FIG. 1A taken at line x-x in FIG. 1A and depicting an embodiment with the internal port formed with a set of flutes.

FIG. 2 is a schematic illustration of a storage container according to an embodiment.

FIG. 3 is a schematic illustration of a storage container according to an embodiment.

FIG. 4A is a schematic illustration of a storage container, depicting the internal port in a U-shaped configuration according to an embodiment.

FIG. 4B is a schematic illustration of the storage container of FIG. 4A, depicting a gas-permeable portion of the internal port according to an embodiment.

FIG. 5 is a schematic illustration of a storage container according to an embodiment.

FIG. 6 is a side view schematic illustration of a storage container according to an embodiment.

FIG. 7A is a schematic illustration of a storage container according to an embodiment.

FIG. 7B is a schematic illustration of the storage container of FIG. 7A after being filled and sealed according to an embodiment.

### DETAILED DESCRIPTION

The embodiments described herein relate to a storage container that can advantageously be used in a wide variety of materials, transportation, processing and/or implantation operations. In particular, the storage containers described herein can allow for a biological material (e.g., a cellular material or culture, etc.) to be loaded and sealed at the point of loading. The storage containers can range from 0.5 ml to 5 liters.

In addition, the singular forms “a,” “an,” and “the” are intended to include the plural forms as well, unless the context indicates otherwise. The terms “comprises,” “includes,” “has,” and the like specify the presence of stated features, steps, operations, elements, components, etc. but do not preclude the presence or addition of one or more other features, steps, operations, elements, components, or groups.

As used herein, the term “about” when used in connection with a referenced numeric indication means the referenced numeric indication plus or minus up to 10% of that referenced numeric indication. For example, the language “about 50” covers the range of 45 to 55. Similarly, the language “about 5” covers the range of 4.5 to 5.5.

As used herein, the term “biologic material” refers to any material that is produced or derived from a living (or recently living) organism. Biologic materials can include, for example, tissue specimens, tissue grafts, cells, blood, or other bodily fluids. Biologic materials can also include plants, plant products, micoorganisms, genetically modified organisms (including cells and cell lines). Biologic materials can also include DNA or RNA (including plasmids, oligonucleotides, cDNA) or viral vectors. Biologic materials can also include material that is produced by a living (or recently living) organism, such as small or large molecule pharmaceuticals.

As used herein, the term “tissue specimen” or “tissue graft” refers to any material that can be used in a tissue repair procedure or other procedures which use tissue grafts (e.g., birth tissue used as patch for healing then removed). Thus, a tissue specimen or a tissue graft can include any of a skin graft, bone tissue, fiber tissue (e.g., tendon tissue, ligament tissue, or the like), ocular tissue (e.g., corneal implants), birth tissue (e.g., amnion graft), cardiovascular tissue (e.g., heart valve) or the like. A tissue specimen or a tissue graft can include a portion of tissue harvested from a donor or a structure component that includes both tissue and non-tissue material (e.g., a synthetic matrix that includes tissue therein). For example, a tissue specimen or a tissue graft can include bone tissue that also includes bone cement or other non-tissue components. As another example, a tissue specimen or tissue graft can include bone chips including cortical bone chips, cancellous bone chips, and corticocancellous bone chips, and/or bone chips with viable bone lineage committed cells.

As used herein, the term “stiffness” relates to an object's resistance to deflection, deformation, and/or displacement produced by an applied force, and is generally understood to be the opposite of the object's “flexibility.” For example, a layer or structure of a container with greater stiffness is more resistant to deflection, deformation and/or displacement when exposed to a force than is a layer or structure of the container having a lower stiffness. Similarly stated, a container (or layer) having a higher stiffness can be characterized as being more rigid than a container (or layer) having a lower stiffness. Stiffness can be characterized in terms of the amount of force applied to the object and the resulting distance through which a first portion of the object deflects, deforms, and/or displaces with respect to a second portion of the object. When characterizing the stiffness of an object, the deflected distance may be measured as the deflection of the portion of the object different than the portion of the object to which the force is directly applied. Said another way, in some objects, the point of deflection is distinct from the point where the force is applied.

Stiffness (and therefore, flexibility) is an extensive property of the object being described, and thus is dependent upon the material from which the object is formed as well as certain physical characteristics of the object (e.g., cross-sectional shape, thickness, boundary conditions, etc.). For example, the stiffness of an object can be increased or decreased by selectively including in the object a material having a desired modulus of elasticity, flexural modulus and/or hardness. The modulus of elasticity is an intensive property of (i.e., is intrinsic to) the constituent material and describes an object's tendency to elastically (i.e., non-permanently) deform in response to an applied force. A material having a high modulus of elasticity will not deflect as much as a material having a low modulus of elasticity in the presence of an equally applied stress. Thus, the stiffness of the object can be decreased, for example, by introducing into the object and/or constructing the object of a material having a relatively low modulus of elasticity. Similarly, the flexural modulus is used to describe the ratio of an applied stress on an object in flexure to the corresponding strain in the outermost portions of the object. The flexural modulus, rather than the modulus of elasticity, is often used to characterize certain materials, for example plastics, that do not have material properties that are substantially linear over a range of conditions. An object with a first flexural modulus is more elastic and has a lower strain on the outermost portions of the object than an object with a second flexural modulus greater than the first flexural modulus. Thus, the stiffness of an object can be reduced by including in the object a material having a relatively low flexural modulus.

Moreover, the stiffness (and therefore flexibility) of an object constructed from a polymer can be influenced, for example, by the chemical constituents and/or arrangement of the monomers within the polymer. For example, the stiffness of an object can be reduced by decreasing a chain length and/or the number of branches within the polymer. The stiffness of an object can also be reduced by including plasticizers within the polymer, which produces gaps between the polymer chains.

The stiffness of an object can also be increased or decreased by changing a physical characteristic of the object, such as the shape or cross-sectional area of the object. For example, an object having a length and a cross-sectional area may have a greater stiffness than an object having an identical length but a smaller cross-sectional area. As another example, the stiffness of an object can be reduced by including one or more stress concentration risers (or discontinuous boundaries) that cause deformation to occur under a lower stress and/or at a particular location of the object. Thus, the stiffness of the object can be decreased by decreasing and/or changing the shape of the object.

As used in this specification, specific words chosen to describe one or more embodiments and optional elements or features are not intended to limit the invention. For example, spatially relative terms-such as “beneath,” “below,” “lower,” “above,” “upper,” “proximal,” “distal,” and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various spatial device positions and orientations.

Similarly, geometric terms, such as “parallel,” “perpendicular,” “round,” or “square” are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

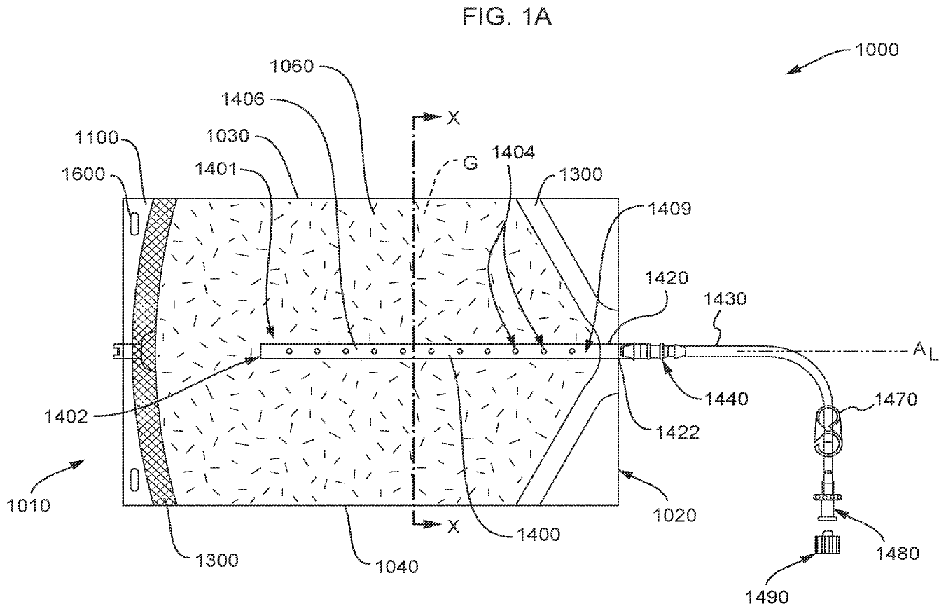

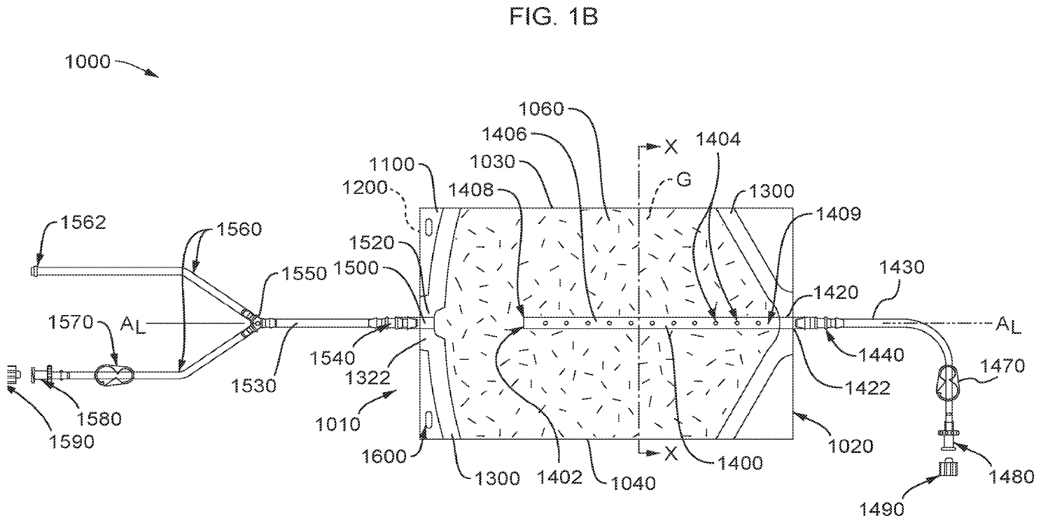

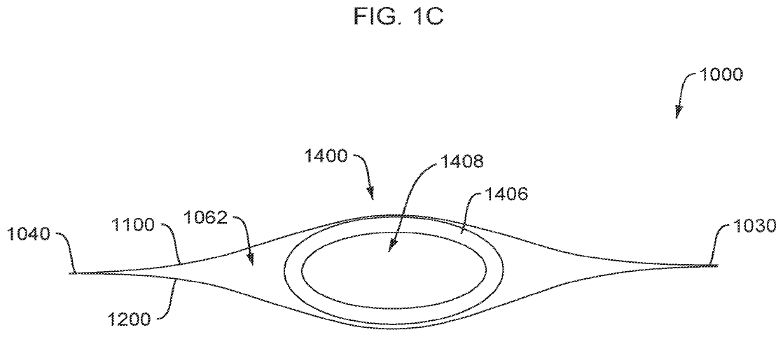

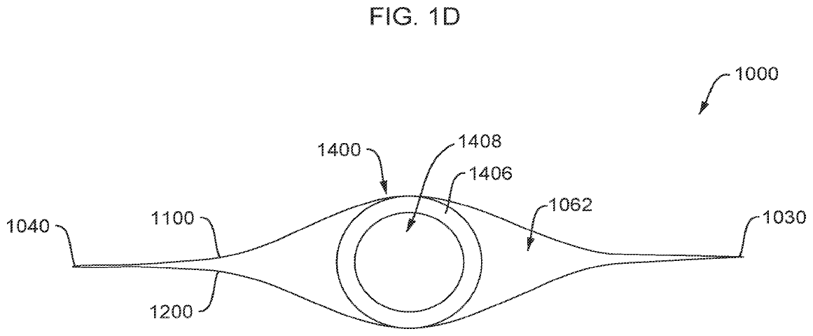

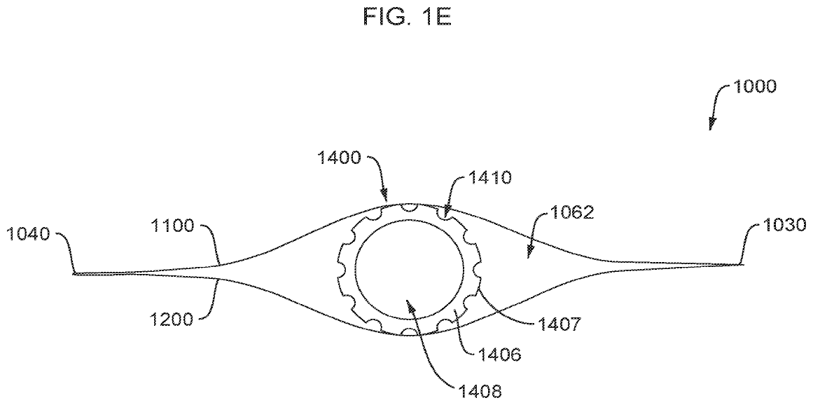

The flexible container 1000 is shown in FIGS. 1A-1D as a container assembly. The container assembly 1000 (and any of the container assemblies described herein) can be used to store any of the biological materials described herein. As described herein, the container assembly 1000 provides a container that can be used for storage, transport, and processing of biological materials G including, for example, cellular matter. It should be appreciated that the elements and features described herein with reference to the flexible container 1000 (e.g., the container assembly) can be combined with elements and features described herein with reference to any other container assembly described herein, and vice-versa. Thus, the shape, size, and features of the internal port 1400 described with reference to the flexible container 1000 are applicable to and can be included in any of the other flexible containers described herein, including the flexible container 2000, the flexible container 3000, the flexible container 4000, the flexible container 5000, the flexible container 6000, and the flexible container 7000.

The flexible container 1000 is constructed from a first layer 1100 and a second layer 1200 and an internal port 1400. The first layer 1100 and the second layer 1200 are coupled together to define a storage volume 1060. The internal port 1400 extends into the storage volume 1060 between first layer 1100 and the second layer 1200. The internal port 1400 maintains a separation volume 1062 (see FIG. 1C) between the first layer 1100 and the second layer 1200. Said another way, the internal port has a thickness that maintains a portion of the first layer 1100 at a distance from the second layer 1200 thereby precluding an adherence of the portion of the first layer 1100 to the second layer 1200. It should be appreciated that the adherence of the first layer 1100 to the second layer 1200 in the defined storage volume 1060 can hinder the introduction of the biological material G into the storage volume 1060.

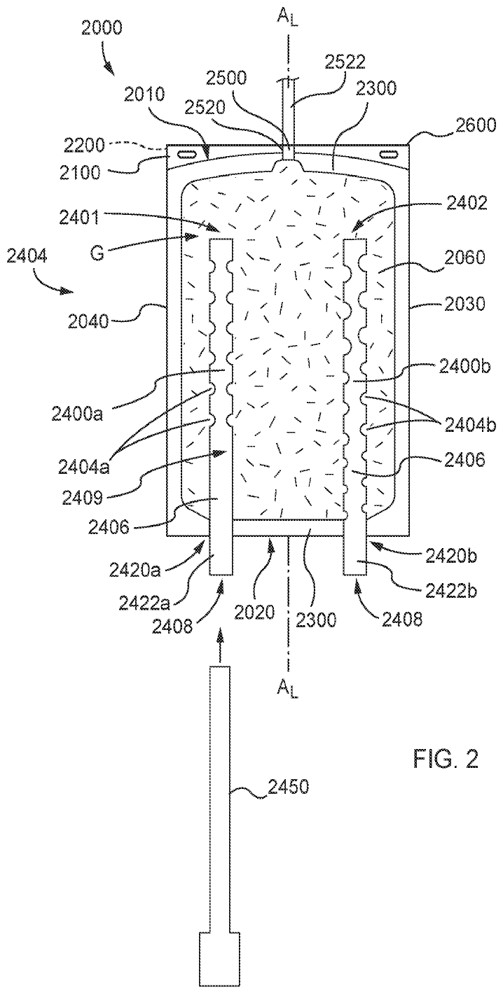

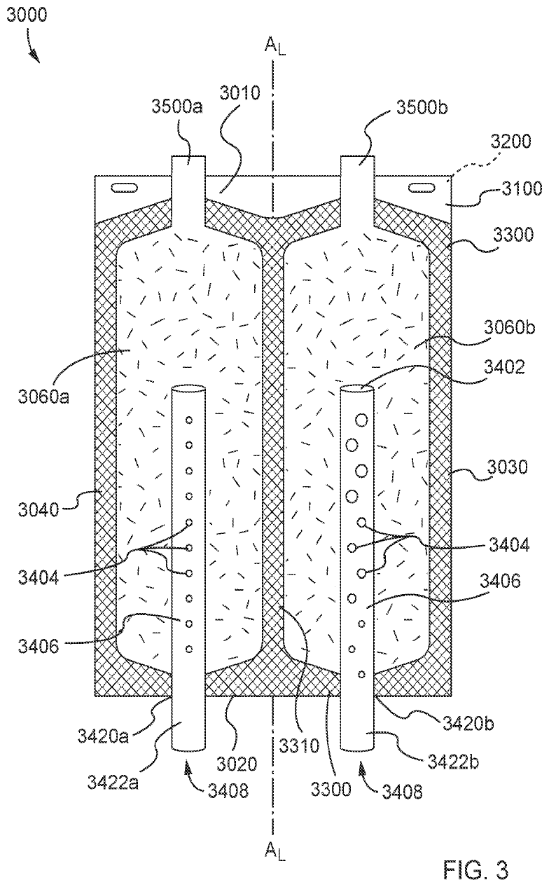

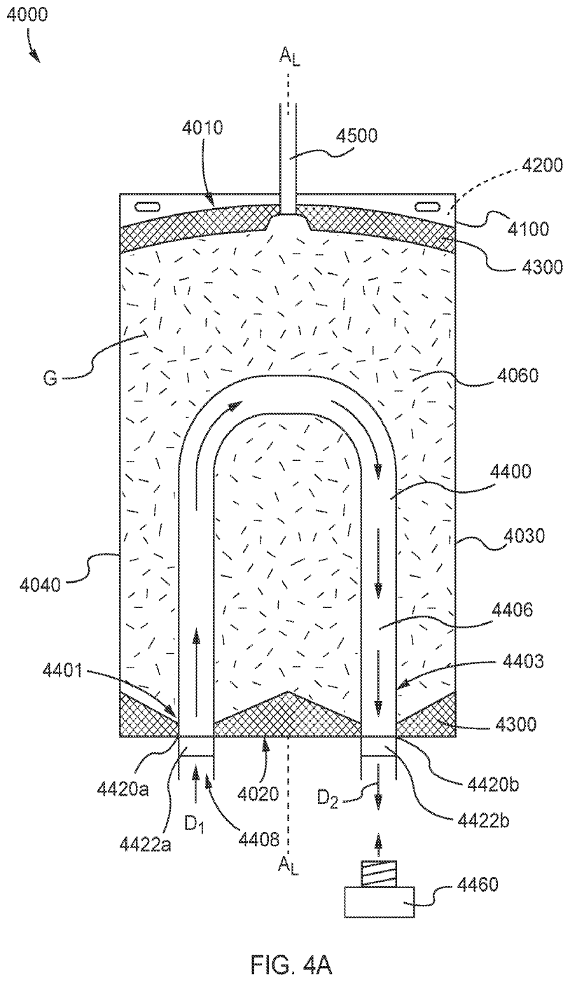

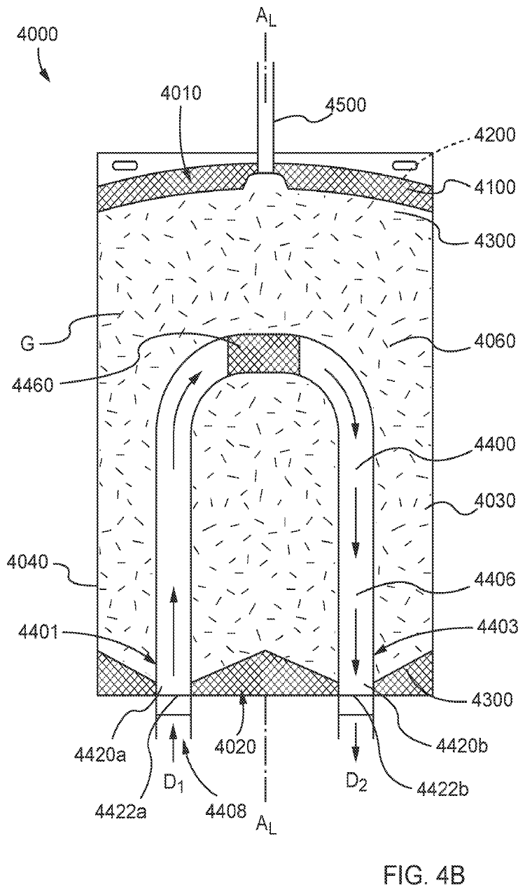

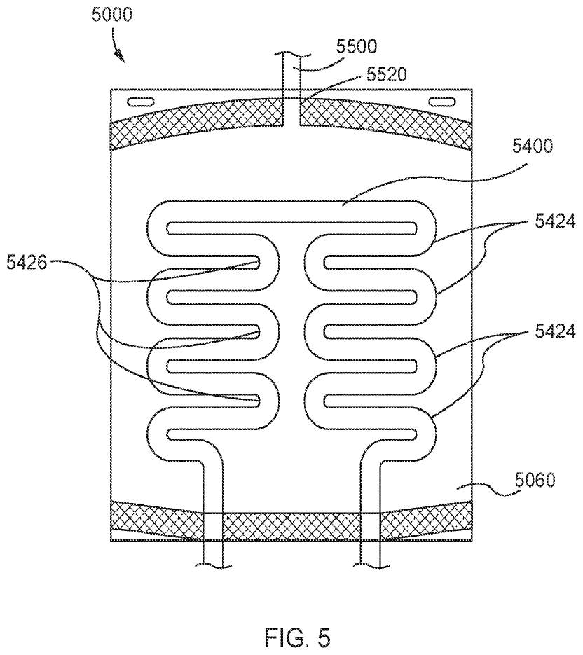

The first layer 1100 and the second layer 1200 include one or more seals 1300 connecting the first layer 1100 and the second layer 1200. The one or more seals 1300 form end seals defining the storage volume 1060. The end seals define a first end portion 1010 and a second end portion 1020. The flexible container 1000 defines a longitudinal axis A_{L} that extends longitudinally from the first end portion 1010 to the second end portion 1020. The flexible container can also include a pair of side edges 1030, 1040 between the first end portion 1010 and the second end portion 1020. The two layers 1100, 1200 can be joined together at the first and second end portions 1010, 1020 and along the side edges 1030, 1040 by any suitable mechanism. The mechanism for connection of the end portions and the side portions can be the same in some embodiments and different in other embodiments. In some embodiments, the connections at the first and second end portions 1010, 1020 can be defined by seals 1300 that are formed by heat bonding or by an adhesive. The side edges can be connected differently. For example, as shown in FIG. 1B (see also FIGS. 4A and 5), the connections at the longitudinal edges 1030, 1040 can be defined by the utilization of a tubular material (e.g., layflat tubular film) flattened out with the edges of the flattened tube forming the longitudinal edges 1030, 1040. However, in other examples, the side edges can be connected similarly to the end portions. For example, the connections at the side edges 1030, 1040 can be defined by seals 1300 that are formed by heat bonding or by an adhesive similar to the end seals (see for example FIGS. 2 and 3).

The materials from which the first layer 1100 and the second layer 1200 are selected to ensure that the two layers can be joined to form the storage volume 1060 within which the biological material G (or any other stored product described herein) is stored while also retaining the desired flexibility. Accordingly, the first and second layers 1100, 1200, respectively, can be constructed of any suitable material. In some embodiments, the layers 1100, 1200 can be constructed from the same material. In some embodiments, the layers 1100, 1200 can be constructed from a different material. The first layer 1100 and/or the second layer can have any suitable thickness or dimensions to provide the desired strength, flexibility, sealing characteristics, or gas permeability. For example, in some embodiments, the first layer 1100 can be between about 1 micron (0.001 mm) and about 3000 microns (3.0 mm). In some embodiments, the first layer 1100 can be between about 1.5 microns (0.00015 mm) and about 200 microns (0.200 mm). In some embodiments, the first layer can be about 1.5 microns (0.00015 mm). The second layer 1200 can have any suitable thickness to provide the desired strength, flexibility, and sealing characteristics. For example, in some embodiments, the first layer 1100 can be between about 1 micron (0.001 mm) and about 3000 microns (3.0 mm). In some embodiments, the first layer 1100 can be between about 1.5 microns (0.00015 mm) and about 200 microns (0.200 mm). In some embodiments, the first layer can be about 1.5 microns (0.00015 mm).

In some embodiments, the layers 1100, 1200 of the container assembly 1000 (or the material from which any of the container assemblies described herein is constructed) can be produced out of any suitable material. In some embodiments, the material can include one or more gas-permeable materials such as fluorinated ethylene propylene (FEP) or polyolefin blends. In some embodiments, the material can include one or more of the following materials: polyethylene (PE), low density polyethylene (LDPE), composites of LDPE, linear low-density polyethylene (LLDPE), high density polyethylene (HDPE), polychlorotrifluoroethylene (PCTFE), ethylene tetrafluoroethylene (ETFE), polytetrafluoroethylene (PTFE), polyurethane, polyimides (coats or non-coated), polyvinyl chloride (PVC), perfluoroalkoxy alkane (PFA), ethylene-vinyl acetate (EVA), polyvinylidene fluoride or polyvinylidene difluoride (PVDF), THV (a polymer of tetrafluoroethylene, hexafluoropropylene and vinylidene fluoride), PFE (Poly (fluorenylene ethynylene)), nylon, and/or composite of nylon. In some embodiments, either or both of the layers 1100, 1200 can be co-extruded and/or laminated with any of the above materials. In some embodiments the material can be rigid. In other embodiments the material can be a laminate combination of rigid and flexible. In other words, at least one of the first layer 1100 or the second layer 1200 can be a support structure. The support structure can be configured to support a biological material G in the storage volume 1060.

As discussed above, the flexible container 1000 includes the internal port 1400. The internal port 1400 includes a structure that extends from the opening 1420 (e.g., a first opening, also referred to as a first container opening) into the storage volume 1060. In other embodiments, the internal port 1400 extends through the seal 1300 defining the opening 1420 and into the storage volume 1060 (i.e., a portion of the internal port 1400 extends into the opening 1420 or even extends outside of the flexible container 1000). The internal port 1400 can be coupled to an external fitting 1422 with the external fitting 1422 being outside of the storage volume 1060 and the internal port 1400 being disposed within the storage volume 1060. In some embodiments, the internal port 1400 and the external fitting 1422 are a contiguous component. In some embodiments, the internal port 1400 and the external fitting 1422 are monolithically constructed. Either one or both of the internal port 1400 and the external fitting 1422 can be connected to or sealed to the layers 1100 and 1200 at the opening 1420. In some embodiments, the internal port 1400 can be positioned within the storage volume 1060 and coupled to or integrated with (e.g., as a laminate layer) the second layer 1200. Coupling the internal port 1400 to the second layer 1200 can facilitate maintaining the internal port 1400 in a desired position (e.g., centered, offset, longitudinally aligned, or other desired position) and/or configuration (e.g., linear, U-shaped, serpentine, or other desired configuration) within the storage volume 1060.

In some embodiments, the internal port 1400 allows biological material to flow into the storage volume 1060 at lower pressures than would be required in the absence of the internal port 1400. For example, traditional methods of conveying biological material into a container can include pressurizing the flow of biological material into the container enough to separate layers of material that may be in close proximity to each other or that may naturally stick together. By introducing the biological material via an internal port 1400 into the separation volume 1062 maintained by the internal port 1400, the remaining separation can be accomplished at reduced pressures compared to a system without the separation volume 1062 maintained and/or established via the internal port 1400.

In some embodiments, the internal port 1400 includes a tube formed by a wall 1406 that defines a hollow interior passage 1408 suitable for passage of the biological material G. Said another way, in some embodiments, the interior passage 1408 has a cross-sectional area sized to facilitate passage of the biological material G. In some embodiments, the internal port 1400 can have a fixed cross-sectional shape. However, in some embodiments, the internal port 1400 can have different cross-sectional shapes depending on conditions affecting the internal port 1400 and/or the storage volume 1060. For example, FIG. 1C depicts the internal port 1400 in a first configuration having a first cross-sectional shape (e.g., an oval or a flattened cross-sectional shape). In some embodiments, the internal port 1400 has the first cross-sectional shape on a condition that the storage volume 1060, the internal port 1400, or both of the storage volume 1060 and the internal port 1400 do not contain the biological material. The internal port 1400 can be configured to transition to a second cross-sectional shape (such as depicted in FIG. 10) to facilitate delivery of the biological material or a treatment material to the storage volume 1060. Said another way, the internal port 1400 can be configured to have the second cross-sectional shape on a condition that the storage volume 1060, the internal port 1400, or both of the storage volume 1060 and the internal port 1400 contain the biological material G.

In some embodiments, the internal port 1400 is at least partially formed from a shape memory material. Accordingly, the internal port 1400 can have a first shape (e.g., the cross-sectional shape depicted in FIG. 1C) in response to a first stimulus and a second shape (e.g., the cross-sectional shape depicted in FIG. 1D) in response to a second stimulus. For example, in some embodiments, the internal port is configured to have a first shape at a first temperature and a second shape, which is different than the first shape, at a second temperature that is different from the first temperature.

In some embodiments, the wall 1406 can have a generally cylindrical cross section, such as depicted in FIG. 1D. In some embodiments, the wall 1406 can include at least one protrusion or recess positioned to facilitate the maintenance of the separation volume 1062. For example, as depicted in FIG. 1E, in some embodiments, the wall 1406 can be formed with a set of flutes 1410 distributed circumferentially about an outer surface 1407 of the internal port 1400. The set of flutes 1410 form channels that facilitate the maintenance of the separation volume 1062.

In accordance with some embodiments, the internal port 1400 includes one or more apertures (e.g., 1402, 1404) which open into the storage volume 1060. In one example, the only fluid communication between the internal passage 1408 and the storage volume 1060 is a single aperture 1402 configured as an end opening. In another example, a set of apertures 1404 extend through the wall 1406 along a portion of the length of the internal port 1400. In some embodiments, the apertures 1404 are arranged such that a portion of the separation volume 1062 is between the apertures 1404 and the first layer 1100 and/or the second layer 1200 and the apertures 1404 are not occluded by the layer 1100, 1200. For example, in some embodiments (see e.g., FIG. 2), the apertures 1404 are positioned orthogonally to a plane that is substantially parallel to the second layer 1200. Having multiple apertures allows for lower resistance for incoming flow of biological material. Having multiple apertures allows for easier ingress or egress of the biological material into or back out of the storage volume 1060. For example, a single hole at the end of the internal port 1400 would make it more difficult to direct the biological material to the single aperture and into the internal port 1400 for egress out of the storage volume 1060. In some embodiments, the apertures 1404 can range from 0.1 millimeters in diameter to 2 cm in diameter.

In some embodiments, the set of apertures 1404 include a set of aperture pairs (e.g., aperture pairs 2404a as depicted in FIG. 2). Each aperture pair includes two opposing apertures that are axially aligned at a single longitudinal position. The aperture pairs are distributed longitudinally along the internal port 1400. Said another way, the apertures 1404 can be through-holes extending through the wall 1406. In one example of such an embodiment, the through-holes can extend through the wall 1406, across the passageway, and out the wall 1406 again. In this manner, there are two opposing holes at each longitudinal position along the internal port 1400.

In some embodiments, each aperture is arranged as a single aperture (e.g., apertures 2404b as depicted in FIG. 2). Each aperture can be axially misaligned with the remaining apertures. In some embodiments, the apertures are distributed longitudinally along the internal port 1400. In some embodiments, the apertures can also be distributed circumferentially about the internal port.

In some embodiments, the apertures 1404 can have different sizes or diameters (e.g., apertures 2404b as depicted in FIG. 2). The arrangement of the apertures 1404 of differing diameters can be based on a desired flow characteristic. For example, in some embodiments, the apertures 1404 positioned at a first distance relative to the opening 1420 can have a first diameter, while apertures 1404 positioned at a greater distance from the opening 1420 can have a second diameter that is greater than the first diameter (see e.g., apertures 2404b in FIG. 2). The apertures with the greater diameter offer less resistance to the passage of a fluid than the apertures with the smaller diameter. Accordingly, such a structure can facilitate the flow of the biological material G into the storage volume 1060 along the length of the internal port 1400 by reducing a resistance gradient along the length of the internal port. However, in some embodiments, the second diameter can be less than the first diameter, which can facilitate removal of the biological material G from the storage volume 1060.

In some embodiments, the apertures 1404 can be offset away from the opening 1420 and the seal 1300, allowing the flow of biological material G into the storage volume 1060 to occur where the separation volume between the layer 1100 and 1200 is greater than that which is produced near the seal 1300. For example, in some embodiments, the wall 1406 of the internal port 1400 defines an uninterrupted portion 1409 adjacent the opening 1420. In such embodiments, the apertures 1404 are positioned between the uninterrupted portion 1409 and an end 1401 of the internal port 1400 within the storage volume 1060.

In other embodiments, at least one of the apertures 1404 can overlap with seal 1300. Similarly stated, the center of at least one aperture can be spaced apart from the seal by less than one half the diameter of the aperture. In such a structure, at least one aperture 1404 is closely positioned relative to the seal 1300, allowing the storage volume 1060 to be able to more completely empty into the internal port than if the apertures 1404 were positioned away from the seal 1300. In this manner, waste can be minimized, and costs can be reduced, especially in light of the high cost of some biological material such as cellular material. In some embodiments, portions of the length of the internal port 1400 can be color-coded or graduated marking can be added, allowing for a user to easily identify the amount of the storage volume 1060 that is filled or emptied. In some embodiments, specific apertures 1404 can be color-coded or have graduated marking, thereby utilizing the apertures 1404 as markers of storage volume 1060 fill.

In some embodiments, the flexible container 1000 can include a sleeve (e.g., sleeve 2450 as depicted in FIG. 2) that can be placed into the internal port 1400 to block or cover the apertures 1404. The sleeve can be configured to transition the internal port 1400 from a first transmission state to a second transmission state. For example, in the first transmission state, the internal port 1400 can be configured to deliver the biological material G into the storage volume 1060 (e.g., via the apertures), while in the second transmission state, the internal port can be configured to convey a treatment media. In some embodiments, the sleeve is gas permeable and impermeable to the biological material G. However, in some embodiments, the sleeve is impermeable to both a gas and the biological material. In this manner the internal port 1400 can also function as a flow element that is fluidically isolated from the storage volume 1060. In such embodiments, a heat exchange fluid (e.g., liquid or gas) can be flowed through the internal port 1400 to facilitate heat transfer to (or from) the material G within the container. In further embodiments, the sleeve can include apertures having a smaller diameter than the apertures of the internal port 1400 and can, therefore, be used to reduce a flow rate into or out of the internal port 1400.

In some embodiments, the internal port 1400 includes a structure that is more rigid than the layers 1100 and/or 1200. As indicated above, the container 1000 is a flexible container with the layers 1100 and 1200 able to collapse together and even stick together. The internal port 1400 includes a stable structure suitable to hold the layers apart, at least in the proximity of the internal structure. In some embodiments, the internal port 1400 is of a different material than that of the layers 1100 and 1200. In some embodiments, the internal port 1400 is of the same material as the layers 1100 and 1200 but includes a different thickness or structure such that it maintains a cross-sectional shape.

In some embodiments, the internal port 1400 has a length that is less than a maximal longitudinal length of the storage volume 1060. In some embodiments, the length of the internal port 1400 relative to the maximal longitudinal length of the storage volume 1060 can vary. In some embodiments, the length of the internal port 1400 is between about 25% and about 95% of the maximal longitudinal length of the storage volume 1060. In one embodiment, the length of the internal port 1400 is about 75% of the maximal longitudinal length of the storage volume 1060.

As indicated above, the flexible container 1000 can include an external fitting 1422. The external fitting 1422 can include one or more of a connector 1440 (e.g., a barbed connector, male luer, female luer, etc.), tubing 1430 (e.g., sterile dockable tubing), a flow control 1470 (e.g., a pinch clamp), a connector 1480 (e.g., a barbed connector, male luer, female luer, etc.), a connector 1490 (e.g., a barbed connector, male luer, female luer, etc.), or other components suited to add biological material (e.g., cellular material) or remove biological material from the flexible container 1000.

As discussed above, the flexible container 1000 includes at least a first opening 1420 suitable for one or more of entry and egress of a biological material into or out of the flexible container 1000. As shown in FIG. 1B, in some embodiments, the flexible container 1000 includes an external port 1500. The external port 1500 can define an opening 1520 (e.g., a second opening) through the seal 1300 on the first end portion 1010 of the flexible container 1000. In some embodiments, the first opening 1420 and the second opening 1520 are axially aligned. The external port 1500 can include an external fitting 1522. The external fitting 1522 can include one or more of a connector 1540 (e.g., a barbed connector, male luer, female luer, etc.), tubing 1530 (e.g., sterile dockable tubing), a flow control 1570 (e.g., a pinch clamp), a connector 1580 (e.g., a barbed connector, male luer, female luer, etc.), a connector 1590 (e.g., a barbed connector, male luer, female luer, etc.), flow control 1570 (e.g., a barbed y connector) or other components suited to remove biological material (e.g., cellular material) from the flexible container 1000. In other embodiments, however, the flexible container 1000 does not include the external port 1500. In such embodiments, the flow into and out of the flexible container 1000 is only through the internal port 1400.

In accordance with some embodiments, the storage volume 1060 includes tapered ends defined by seals 1300. Each of the tapered seal ends can extend to opening 1420 or opening 1520. The taper allows for the biological material contained in the storage volume 1060 to be funneled toward an opening (e.g., 1420 or 1520). It is, however, appreciated that other structures and shapes are also contemplated herein. The end seal 1300 at 1020 can include a chevron shape that defines a taper to the internal port 1400. The seal can be flat with separate sides being 180 degrees to a chevron shape with an angle of 15 degrees between the separate sides of the seal. The seal can also include all angles between 180 degrees and 15 degrees.

In some embodiments, the flexible container 1000 can include other structural components. For example, the flexible container 1000 can include apertures 1600 suitable to work with hangers on medical stands.

In accordance with some embodiments, a flexible container 2000 can include a set of internal ports as shown in FIG. 2. The container assembly 2000 (and any of the container assemblies described herein) can be used to store any of the biological materials described herein. As described herein, the container assembly 2000 provides a container that can be used for storage, transport, and processing of biological materials G including, for example, cellular matter. It should be appreciated that the elements and features described herein with reference to the flexible container 2000 (e.g., the container assembly) can be combined with elements and features described herein with reference to any other container assembly described herein, and vice-versa. Thus, the shape, size, and features of the internal ports 2400a, 2400b described with reference to the flexible container 2000 are applicable to and can be included in any of the other flexible containers described herein, including the flexible container 1000, the flexible container 3000, the flexible container 4000, the flexible container 5000, the flexible container 6000, and the flexible container 7000.

The flexible container 2000 is constructed from a first layer 2100 and a second layer 2200 and a set of internal ports 2400a and 2400b. The first layer 2100 and the second layer 2200 are coupled together to define a storage volume 2060. The internal ports 2400a, 2400b extend into the storage volume 2060 between first layer 2100 and the second layer 2200. The first layer 2100 and the second layer 2200 include one or more seals 2300 connecting the first layer 2100 and the second layer 2200. The one or more seals 2300 form end seals defining the storage volume 2060. The end seals define a first end portion 2010 and a second end portion 2020. The flexible container 2000 defines a longitudinal axis A_{L} that extends longitudinally from the first end portion 2010 to the second end portion 2020. The flexible container can also include a pair of side edges 2030, 2040 between the first end portion 2010 and the second end portion 2020. The two layers 2100, 2200 can be joined together at the first and second end portions 2010, 2020 and along the side edges 2030, 2040 by any suitable mechanism. The mechanism for connection of the end portions and the side portions can be the same in some embodiments and different in other embodiments. For example, the connections at the first and second end portions 2010, 2020 can be defined by seals 2300 that are formed by heat bonding or by an adhesive. The connections at the longitudinal edges (i.e., side edges) 2030, 2040 can be similarly connected as shown. In other embodiments, the longitudinal edges 2030 and 2040 can be defined by the utilization of a tubular material (e.g., layflat tubular film) flattened out with the edges of the flattened tube forming the longitudinal edges 2030, 2040 (See e.g., FIGS. 1A, 4A, and 5).

The materials from which the first layer 2100 and the second layer 2200 are selected to ensure that the two layers can be joined to form the storage volume 2060 within which the biological material G (or any other stored product described herein) is stored while also retaining the desired flexibility. Accordingly, the first and second layers 2100, 2200, respectively, can be constructed of any suitable material. The first layer 2100 and/or the second layer can have any suitable thickness or dimensions to provide the desired strength, flexibility, sealing characteristics, or gas permeability such as those discussed above with respect to the flexible containers 1000 and 2000. In some embodiments, the layers 2100, 2200 of the container assembly 2000 (or the material of any of the container assemblies described herein) can be produced out of any suitable material. For example, the materials can be those discussed above with regard to the flexible container 1000. In some embodiments, either one or both of the layers 2100, 2200 can be co-extruded and/or laminated with any of the above materials.

As discussed above, the flexible container 2000 includes a set of internal ports 2400a and 2400b. The internal ports 2400a and 2400b can include structures that extend from openings 2420a 2420b into the storage volume 2060. In other embodiments, the internal ports 2400a and 2400b extend through the seal 2300 defining the openings 2420a 2420b and into the storage volume 2060. The internal ports 2400a and 2400b can be coupled to external fittings 2422a and 2422b with the external fittings 2422a and 2422b being outside of the storage volume 2060 and the internal ports 2400a and 2400b being disposed within the storage volume 2060. In some embodiments, the internal ports 2400a and 2400b and the external fittings 2422a and 2422b are a contiguous component. Either or both of the internal ports 2400a and 2400b and the external fittings 2422a and 2422b can be connected to or sealed to the layers 2100 and 2200 at the opening 2420.

In some embodiments, the internal ports 2400a and 2400b allow biological material to flow into the storage volume 2060 at lower pressures than would be required in the absence of the internal ports 2400a and 2400b. For example, traditional methods of conveying biological material into a container can include pressurizing the flow of biological material into the container enough to separate layers of material that may be in close proximity to each other or that may naturally stick together. By introducing the biological material via an internal ports 2400a and 2400b, the layers are already partially separated due to the internal ports 2400a and 2400b, and creating the remaining separation is accomplished at reduced pressures compared to a system without the internal ports 2400a and 2400b.

The internal ports 2400a and 2400b can include a similar structure to the internal port 1400 discussed above with the internal ports 2400a and 2400b having a wall 2406 that defines an interior passage 2408 and one or more apertures (e.g., 2402, 2404) open into the storage volume 2060. Similarly, each of these component parts of the internal port can be similar to those discussed above with regard to the flexible container 1000. In some embodiments, the flexible container 2000 can include other structural components. For example, the flexible container 2000 can include apertures 2600 suitable to work with hangers on medical stands. In some embodiments, the flexible container 2000 includes an external port 2500. The external port 2500 can define an opening 2520 through the seal 2300 on the first end portion 2010 of the flexible container 2000.

As depicted in FIG. 2, in some embodiments, the set of apertures 2404 includes a set of aperture pairs 2404a. Each aperture pair 2404a includes two opposing apertures that are axially aligned at a single longitudinal position. The aperture pairs 2404a are distributed longitudinally along the internal port 2400a. Said another way, the apertures 2404a can be through-holes extending through the wall 2406. In one example of such an embodiment, the through-holes can extend through the wall 2406, across the passageway, and out the wall 2406 again. In this manner, there are two opposing holes at each longitudinal position along the internal port 2400.

In some embodiments, each aperture is arranged as a single aperture 2404b. Each single aperture 2404b can be axially misaligned with the remaining apertures 2404b. In some embodiments, the apertures 2404b are distributed longitudinally along the internal port 2400. In some embodiments, the apertures 2404b can also be distributed circumferentially about the internal port (e.g., internal port 2400b).

In some embodiments, the apertures can be offset away from the opening 2420 and the seal 2300, allowing the flow of biological material G into the storage volume 2060 to occur where the separation volume between the layer 2100 and 2200 is greater than that which is produced near the seal 2300. For example, in some embodiments, the wall 2406 of the internal port 2400 defines an uninterrupted portion 2409 adjacent the opening 2420. In such embodiments, the apertures 2404 are positioned between the uninterrupted portion 2409 and an end 2401 of the internal port 2400 within the storage volume 2060.

As depicted by apertures 2404b, in some embodiments, the apertures 2404 can have different diameters. The arrangement of the apertures 2404 of differing diameters can be based on a desired flow characteristic. For example, in some embodiments, such as depicted by apertures 2404b, the apertures positioned at a first distance relative to the opening 2420b can have a first diameter, while apertures positioned at a greater distance from the opening 2420b can have a second diameter that is greater than the first diameter. The apertures with the greater diameter offer less resistance to the passage of a fluid than the apertures with the smaller diameter. Accordingly, such a structure can facilitate the flow of the biological material G into the storage volume 2060 along the length of the internal port 2400 by reducing a resistance gradient along the length of the internal port. However, in some embodiments, the second diameter can be less than the first diameter, which can facilitate removal of the biological material G from the storage volume 2060.

In some embodiments, the flexible container 2000 can include a sleeve 2450 that can be placed into an internal port (e.g., internal port 2400a) to block or cover the apertures 2404. The sleeve 2450 can be configured to transition the internal port from a first transmission state to a second transmission state. For example, in the first transmission state, the internal port can be configured to deliver the biological material G into the storage volume 2060, while in the second transmission state, the internal port can be configured to convey a treatment media. In some embodiments, the sleeve 2450 is gas permeable and impermeable to the biological material G. However, in some embodiments, the sleeve 2450 is impermeable to both gas and the biological material. In this manner the internal port can also function as a flow element that is fluidically isolated from the storage volume 2060. In such embodiments, a heat exchange fluid (e.g., liquid or gas) can be flowed through the internal port to facilitate heat transfer to (or from) the material G within the container. In further embodiments, the sleeve 2450 can include apertures (not shown) having a smaller diameter than the apertures of the internal port and can, therefore, be used to reduce a flow rate into or out of the internal port.

In some embodiments, the internal ports 2400a and 2400b can include the same structure. For example, both internal ports 2400a and 2400b can be used for entry and egress of biological materials (e.g., cellular material). Alternatively, the internal ports 2400a and 2400b can include different structures. For example, one of the internal ports (e.g., 2400a) can be used for entry of biological materials (e.g., cellular material), while the other internal port (e.g., 2400b) can be used for egress of the biological materials. In another example, one of the internal ports (e.g., 2400a) can be used for entry and egress of biological materials (e.g., cellular material), while the other internal port (e.g., 2400b) can be used for treatment of the biological material (e.g., introduction of beneficial gases, introduction of cooling materials, introduction of nutrient base, etc.). Said another way, the first internal port 2400a can be configured to facilitate a first procedure (e.g., delivery of the biological material G into the storage volume 2060). The second internal port 2400b can be configured to facilitate a second procedure (e.g., removal of the biological material G from the storage volume 2060). In some embodiments, the first procedure corresponds to a delivery of the biological material G into the storage volume 2060 and a removal of the biological material G from the storage volume 2060, while the procedure corresponds to a conveying of a treatment media.

In accordance with some embodiments, a flexible container 3000 can include a set of internal ports as shown in FIG. 3. The container assembly 3000 (and any of the container assemblies described herein) can be used to store any of the biological materials described herein. As described herein, the container assembly 3000 provides a container that can be used for storage, transport, and processing of biological materials G including, for example, cellular matter. It should be appreciated that the elements and features described herein with reference to the flexible container 3000 can be combined with elements and features described herein with reference to any other container assembly described herein, and vice-versa. Thus, the shape, size, and features of the internal port 3400 described with reference to the flexible container 3000 are applicable to and can be included in any of the other flexible containers described herein, including the flexible container 1000, the flexible container 2000, the flexible container 4000, the flexible container 5000, the flexible container 6000, and the flexible container 7000.

The flexible container 3000 is constructed from a first layer 3100, a second layer 3200, and a set of internal ports 3400a and 3400b separated by at least a portion of seal 3310 (e.g., a center seal). The first layer 3100 and the second layer 3200 are coupled together to define a storage volume 3060. The internal ports 3400a and 3400b extend into the storage volumes 3060a and 3060b, respectively, between first layer 3100 and the second layer 3200. The first layer 3100 and the second layer 3200 include one or more seals 3300 connecting the first layer 3100 and the second layer 3200. The one or more seals 3300 form end seals defining the storage volumes 3060a and 3060b. The end seals define a first end portion 3010 and a second end portion 3020. The flexible container 3000 defines a longitudinal axis A_{L} that extends longitudinally from the first end portion 3010 to the second end portion 3020. The flexible container can also include a pair of side edges 3030, 3040 between the first end portion 3010 and the second end portion 3020. The two layers 3100, 3200 can be joined together at the first and second end portions 3010, 3020 and along the side edges 3030, 3040 by any suitable mechanism. The mechanism for connection of the end portions and the side portions can be the same in some embodiments and different in other embodiments. For example, the connections at the first and second end portions 3010, 3020 can be defined by seals 3300 that are formed by heat bonding or by an adhesive. The connections at the longitudinal edges 2030, 2040 can be similarly connected as shown. In other embodiments, the longitudinal edges 2030 and 2040 can be defined by the utilization of a tubular material (e.g., layflat tubular film) flattened out with the edges of the flattened tube forming the longitudinal edges 2030, 2040 (See e.g., FIGS. 1A, 4A, and 5).

The materials from which the first layer 3100 and the second layer 3200 are selected to ensure that the two layers can be joined to form the storage volumes 3060a and 3060b within which the biological material G (or any other stored product described herein) is stored while also retaining the desired flexibility. Accordingly, the first and second layers 3100, 3200, respectively, can be constructed of any suitable material. The first layer 3100 and/or the second layer can have any suitable thickness or dimensions to provide the desired strength, flexibility, sealing characteristics, or gas permeability such as those discussed above with respect to the flexible container 1000. In some embodiments, the layers 3100, 3200 of the container assembly 3000 (or the material of any of the container assemblies described herein) can be produced out of any suitable material. For example, the materials can be those discussed above with regard to the flexible container 1000. In some embodiments, either or both of the layers 3100, 3200 can be co-extruded and/or laminated with any of the above materials.

As discussed above, the flexible container 3000 includes a set of internal ports 3400a and 3400b. The internal ports 3400a and 3400b can include structures that extend from openings 3420a 3420b into the storage volumes 3060a and 3060b, respectively. In other embodiments, the internal ports 3400a and 3400b extend through the seal 3300 defining the openings 3420a 3420b and into the storage volumes 3060a and 3060b. The internal ports 3400a and 3400b can be coupled to external fittings 3422a and 3422b with the external fittings 3422a and 3422b being outside of the storage volumes 3060a and 3060b and the internal ports 3400a and 3400b being disposed within the storage volumes 3060a and 3060b. In some embodiments, the internal ports 3400a and 3400b and the external fittings 3422a and 3422b are a contiguous component. Either one or both of the internal ports 3400a and 3400b and the external fittings 3422a and 3422b can be connected to or sealed to the layers 3100 and 3200 at the opening 3420.

In some embodiments, the internal ports 3400a and 3400b allow biological material to flow into the storage volumes 3060a and 3060b at lower pressures than would be required in the absence of the internal ports 3400a and 3400b. For example, traditional solutions include pressurizing the flow of biological material into the container enough to separate layers of material that naturally stick together. By introducing the biological material via internal ports 3400a and 3400b, the layers are already partially separated due to the internal ports 3400a and 3400b, and creating the remaining separation is accomplished at reduced pressures compared to a system without the internal ports 3400a and 3400b.

The internal ports 3400a and 3400b can include a similar structure to the internal port 1400 discussed above with the internal ports 3400a and 3400b having a wall 3406, interior passage 3408, and one or more apertures (e.g., 3402, 3404) open into the storage volumes 3060a and 3060b. Similarly, each of these component parts of the internal port can be similar to those discussed above with regard to the flexible container 1000. In some embodiments, the flexible container 3000 can include other structural components. In some embodiments, the flexible container 3000 includes a first external port 3500a and a second external port 3500b. The ports 3500a and 3500b can define openings through the seal 3300 on the first end portion 3010 of the flexible container 3000.

In some embodiments, the internal ports 3400a and 3400b can include the same structure. For example, both internal ports 3400a and 3400b can be used for entry and egress of biological materials (e.g., cellular material). Alternatively, the internal ports 3400a and 3400b can include different structures. For example, one of the internal ports (e.g., 3400a) can be used for entry of biological materials (e.g., cellular material), while the other internal port (e.g., 3400b) can be used for egress of the biological materials. In another example, one of the internal ports (e.g., 3400a) can be used for entry and egress of biological materials (e.g., cellular material), while the other internal port (e.g., 3400b) can be used for treatment of the biological material (e.g., introduction of beneficial gases, introduction of colling materials, introduction of nutrient base, etc.).

In accordance with various embodiments, the center seal 3310 can be a partial seal or a permeable seal (e.g., the seal can have openings extending between volume 3060a and volume 3060b of the containers) allowing flow of biological material between volume 3060a and volume 3060b. With the movement of biological material between volume 3060a and volume 3060b, internal ports 3400a and 3400b can be structured differently and used for different purposes as discussed above with reference to container 2000.

In accordance with some embodiments, a flexible container 4000 can include an internal port as shown in FIGS. 4A and 4B. The container assembly 4000 (and any of the container assemblies described herein) can be used to store any of the biological materials described herein. As described herein, the container assembly 4000 provides a container that can be used for storage, transport, and processing of biological materials G including, for example, cellular matter. It should be appreciated that the elements and features described herein with reference to the flexible container 4000 (e.g., the container assembly) can be combined with elements and features described herein with reference to any other container assembly described herein, and vice-versa. Thus, the shape, size, and features of the internal port 4400 described with reference to the flexible container 4000 are applicable to and can be included in any of the other flexible containers described herein, including the flexible container 1000, the flexible container 2000, the flexible container 3000, the flexible container 5000, the flexible container 6000, and the flexible container 7000.

The flexible container 4000 is constructed from a first layer 4100 and a second layer 4200 and an internal port 4400. The internal port extends between a first end portion 4401 and a second end portion 4403 As shown, the internal port 4400 is routed through the interior of storage volume 4060 allowing it to provide additional processing or treatment to the biological material G stored in the storage volume 4060.

The first layer 4100 and the second layer 4200 are coupled together to define a storage volume 4060. The internal port 4400 extends into the storage volume 4060 between first layer 4100 and the second layer 4200. The first layer 4100 and the second layer 4200 include one or more seals 4300 connecting the first layer 4100 and the second layer 4200. The one or more seals 4300 form end seals defining the storage volume 4060. The end seals define a first end portion 4010 and a second end portion 4020. The flexible container 4000 defines a longitudinal axis A_{L} that extends longitudinally from the first end portion 4010 to the second end portion 4020. The flexible container can also include a pair of side edges 4030, 4040 between the first end portion 4010 and the second end portion 4020. The two layers 4100, 4200 can be joined together at the first and second end portions 4010, 4020 and along the side edges 4030, 4040 by any suitable mechanism. The mechanism for connection of the end portions and the side portions can be the same in some embodiments and different in other embodiments. In some embodiments, the connections at the first and second end portions 4010, 4020 can be defined by seals 4300 that are formed by heat bonding or by an adhesive. The side edges can be connected differently. For example, as shown (see also FIGS. 1B and 5A), the connections at the longitudinal edges 4030, 4040 can be defined by the utilization of a tubular material (e.g., layflat tubular film) flattened out with the edges of the flattened tube forming the longitudinal edges 4030, 4040 (i.e., side edges). However, in other examples, the side edges can be connected similarly to the end portions. For example, the connections at the side edges 4030, 4040 can be defined by seals 4300 that are formed by heat bonding or by an adhesive similar to the end seals (see for example FIGS. 2 and 3).

The materials from which the first layer 4100 and the second layer 4200 are constructed are selected to ensure that the two layers can be joined to form the storage volume 4060 within which the biological material G (or any other stored product described herein) is stored while also retaining the desired flexibility. Accordingly, the first and second layers 4100 and 4200, respectively, can be constructed of any suitable material. The first layer 4100 and/or the second layer can have any suitable thickness or dimensions, such as those discussed above with respect to the flexible containers 1000, 2000, and 3000, to provide the desired strength, flexibility, sealing characteristics, or gas permeability. In some embodiments, the layers 4100, 4200 of the container assembly 4000 (or the material of any of the container assemblies described herein) can be produced out of any suitable material. For example, the materials can be those discussed above with regard to the flexible container 1000, 2000, and 3000. In some embodiments, either or both of the layers 4100, 4200 can be co-extruded and/or laminated with any of the above materials.

As discussed above, the flexible container 4000 includes an internal port 4400. The internal port 4400 can include structures that extend from openings 4420a to 4420b into the storage volume 4060. In other embodiments, the internal port 4400 extends through the seal 4300 defining the openings 4420a and 4420b and into the storage volume 4060. The internal port 4400 can be coupled to external fittings 4422a and 4422b with the external fittings 4422a and 4422b being outside of the storage volume 4060 and the internal port 4400 being disposed within the storage volume 4060. In some embodiments, the internal port 4400 and the external fittings 4422a and 4422b are a contiguous component. The external fittings 4422a and 4422b can be connected to or sealed to the layers 4100 and 4200 at the opening 4420.

In some embodiments, the internal port 4400 allows biological material to flow into the storage volume 4060 at lower pressures than would be required in the absence of the internal port 4400. For example, traditional solutions include pressurizing the flow of biological material into the container enough to separate layers of material that naturally stick together. By introducing the biological material via internal port 4400, the layers are already partially separated due to the internal port 4400 and creating the remaining separation is accomplished at reduced pressures compared to a system without the internal port 4400.

The internal port 4400 can include a similar structure to the internal port 1400 discussed above with the internal port 4400 having a wall 4406, interior passage 4408, and one or more apertures open into the storage volume 4060. Similarly, each of these component parts of the internal port can be similar to those discussed above with regard to the flexible container 1000.

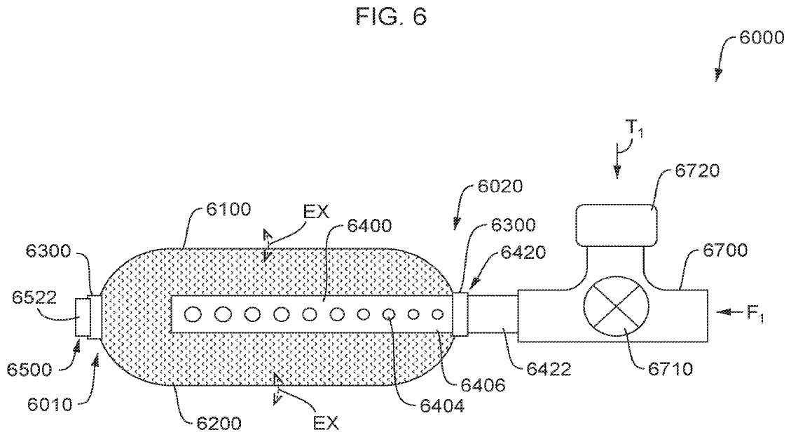

In some embodiments, the internal port 4400 can transport a treatment material through the volume and more specifically through the mass of the biological material. The treatment material could include a treatment gas that aids in the survival of the biological material. The treatment material could include a heat transfer media such as a coolant suitable to cool or cryogenically freeze the biological material. In one example, the treatment material can enter through the external fitting 4422a in the direction D1 and pass through the internal port 4400 and exit through the external fitting 4422b in the direction D2. In embodiments in which the internal port 4400 transports a heat transfer media, the treatment material can just flow through the internal port 4400. In such embodiments, the internal port 4400 can include a conductive material suitable to efficiently transfer energy between the treatment material and the biological material. In embodiments in which the internal port 4400 transports a gas, the internal port 4400 can include a gas-permeable portion 4460 of the wall 4406. The gas-permeable portion 4460, and all gas-permeable structures described herein, can include a plurality of pores or other structures that permit the passage of a molecule in a gaseous state but are impermeable to molecules in a solid or liquid state. In another embodiment, as discussed in other embodiments (e.g., internal port 1400, 2400, and 3400), the internal port included apertures. In an embodiment utilizing the internal port 4400 as a gaseous transport, a sleeve as described herein with reference to sleeve 2450 having a permeable membrane can be included within the internal port 4400 such that apertures in the wall of the internal port 4400 allow gas to pass through the permeable membrane and into biological material. In other embodiments (discussed in more detail below with reference to FIG. 6) gas can reside in the internal port 4400 and gas can pass through an external filter connected with the internal port 4400.

In other embodiments, internal port 4400 can include similar structures to those discussed above with regards to internal port 1400, 2400, and 3400 including apertures along the length. The biological material can be injected into the internal port 4400 and be spread out more uniformly in the storage volume 4060 due to the added length of the connection between the two sides of the internal port 4400. In one example, biological material can be injected into both external fitting 4422a and 4422b allowing the pressure from each side to transport the biological material into the 4030. In another example, one side could be blocked, such as external fitting 4422b could include a removable plug 4460 configured to temporarily occlude an end of the internal port 4400. Biological material would be injected into external fitting 4422a and pass through internal port 4400 but not out external fitting 4422b because of the removable plug 4460. This would cause the biological material to be transported out of the internal port 4400.

In some embodiments the internal port 4600 has an absence of apertures (e.g., is fluidly isolated from the storage volume) and transports treatment material. In such embodiments, the internal port 4400 is positioned internally but is ported to the external environment relative to the container 4000. In this way, the internal port 4400 is a passage positioned within the storage volume 4060. In other words, in some embodiments, the internal port 4400 can convey the treatment through a material G positioned within the storage volume 4060 while precluding contact between the treatment material and the material G.

In some embodiments, the flexible container 4000 includes an external port 4500. The external port 4500 can define an opening 4520 through the seal 4300 on the first end portion 4010 of the flexible container 4000.

In accordance with some embodiments, a flexible container 5000 can include an internal port 5400 as shown in FIG. 5. The flexible container 5000 includes the various embodiments and examples discussed above with respect to flexible container 4000 in FIGS. 4A and 4B. The flexible container 5000 deviates from the disclosure above with respect to the path of the internal port 5400 through the volume 5060. Here the internal port 5400 includes a serpentine path. The serpentine path includes exterior bends 5424 and interior bends 5426 allowing the internal port 5400 to pass through a greater percentage of biological material or volume as compared to the internal ports discussed above (e.g., 1400, 2400, 3400, or 4400). This allows for a greater distribution of treatment material (e.g., gas, heat transfer fluid, etc.) and/or biological material distribution. In such embodiments, the length of the internal port 5400 can be greater than a maximal longitudinal length of the storage volume. For example, the internal port 5400 can have a length that is at least 250% of the maximal longitudinal length of the storage volume. In some embodiments, the flexible container 5000 includes an external port 5500. The external port 5500 can define an opening 5520 through the seal 5300 on the first end 5010 of the flexible container 1000. For the sake of clarity, the description of various optional elements of flexible container 5000 are omitted, but would be consistent with the disclosure of FIGS. 4A and 4B.

In some embodiments, the internal port 5400 is formed, at least in part, from a shape memory material. In such embodiments, the serpentine configuration can be established by a response of the internal port 5400 to a stimulus. For example, in response to a change in temperature, the internal port 5400 can transition from a collapsed configuration to a serpentine configuration.

The flexible container 6000 is shown in FIG. 6 is illustrated as a cross-section side view. While the container 6000 is described separately from containers 1000, 2000, 3000, 4000, and 5000 herein, it should be appreciated that each of the aspects, embodiments, and examples relating to container 6000 can be applied to any of the containers 1000, 2000, 3000, 4000, and 5000. The container assembly 6000 (and any of the container assemblies described herein) can be used to biological materials described herein. As described herein, the container assembly 6000 provides a container that can be used for storage, transport, and processing of biological materials G including, for example, cellular matter.

The flexible container 6000 is constructed from a first layer 6100 and a second layer 6200 and an internal port 6400. The first layer 6100 and the second layer 6200 are coupled together to define a storage volume 6060. The internal port 6400 extends into the storage volume 6060 between first layer 6100 and the second layer 6200. The first layer 6100 and the second layer 6200 include one or more seals 6300 connecting the first layer 6100 and the second layer 6200. The one or more seals 6300 form end seals defining the storage volume 6060. The end seals define a first end portion 6010 and a second end portion 6020. The flexible container 6000 defines a longitudinal axis A_{L} that extends longitudinally from the first end portion 6010 to the second end portion 6020. The flexible container can also include a pair of side edges 6030, 6040 between the first end portion 6010 and the second end portion 6020. The two layers 6100, 6200 can be joined together at the first and second end portions 6010, 6020 and along the side edges (not shown) by any suitable mechanism. The mechanism for connection of the end portions and the side portions can be the same in some embodiments and different in other embodiments. For example, as shown in FIG. 1B (see also FIGS. 4A and 5), the connections at the longitudinal edges (i.e., side edges) 1030, 1040 can be defined by the utilization of a tubular material (e.g., layflat tubular film) flattened out with the edges of the flattened tube forming the longitudinal edges 1030, 1040. However, in other examples, the side edges can be connected similarly to the end portions. For example, the connections at the side edges 1030, 1040 can be defined by seals 1300 that are formed by heat bonding or by an adhesive similar to the end seals (see for example FIGS. 2 and 3).

The materials from which the first layer 6100 and the second layer 6200 are selected to ensure that the two layers can be joined to form the storage volume 6060 within which the biological material G (or any other stored product described herein) is stored while also retaining the desired flexibility. Accordingly, the first and second layers 6100, 6200, respectively, can be constructed of any suitable material. In some embodiments, the layers 6100, 6200 can be constructed from the same material. In some embodiments, the layers 6100, 6200 can be constructed from a different material. The first layer 6100 and/or the 6200 second layer can have any suitable thickness or dimensions to provide the desired strength, flexibility, sealing characteristics, or gas permeability. For example, in some embodiments, the first layer 6100 can be between about 60 microns (0.010 mm) and about 2000 microns (2.0 mm). In some embodiments, the first layer 6100 can be between about 50 microns (0.050 mm) and about 200 microns (0.200 mm). In some embodiments, the first layer can be between about 50 microns (0.050 mm) and about 6000 microns (0.100 mm). The second layer 6200 can have any suitable thickness to provide the desired strength, flexibility, and sealing characteristics. For example, in some embodiments, the first layer 6200 can be between about 60 microns (0.010 mm) and about 2000 microns (2.0 mm). In some embodiments, the second layer 6200 can be between about 50 microns (0.050 mm) and about 200 microns (0.200 mm). In other embodiments, the second layer 6200 can be between about 50 microns (0.050 mm) and about 6000 microns (0.100 mm).

In some embodiments, the layers 6100, 6200 of the container assembly 6000 (or the material of any of the container assemblies described herein) can be produced out of any suitable material. In some embodiments, the material can include one or more gas-permeable materials such as fluorinated ethylene propylene (FEP) or polyolefin blends. In embodiments, in which cellular material is used, gas permeability aids in keeping the cells healthy. Thus, in embodiments in which the internal port 6400 is used to distribute the cellular material into the storage volume 6060, a gas-permeable material helps keep the cells alive. Furthermore, usage of the internal port 6400 helps separate gas permeable layers (e.g., FEP layers) which have a tendency to stick together. This also reduces the trauma to the cells when distributed into the storage volume 6060.

In embodiments, when gas permeability is not called for, other suitable materials can also be used. In some of these embodiments, the internal port 6400 might be used to provide internal gas transfer. In some of these embodiments, the internal port 6400 might be used to circulate heat transfer fluid to the storage volume 6060. Such materials can include one or more of the following materials: polyethylene (PE), low density polyethylene (LDPE), composites of LDPE, linear low-density polyethylene (LLDPE), high density polyethylene (HDPE), polychlorotrifluoroethylene (PCTFE), ethylene tetrafluoroethylene (ETFE), polytetrafluoroethylene (PTFE), polyurethane, polyimides (coats or non-coated), polyvinyl chloride (PVC), perfluoroalkoxy alkane (PFA), ethylene-vinyl acetate (EVA), polyvinylidene fluoride or polyvinylidene difluoride (PVDF), THV (a polymer of tetrafluoroethylene, hexafluoropropylene and vinylidene fluoride), PFE (Poly (fluorenylene ethynylene)), nylon, and/or composite of nylon. In some embodiments, either or both of the layers 6100, 6200 can be co-extruded and/or laminated with any of the above materials.

As discussed above, the flexible container 6000 includes the internal port 6400. The internal port 6400 includes a structure that extends from the opening 6420 into the storage volume 6060. In other embodiments, the internal port 6400 extends through the seal 6300 defining the opening 6420 and into the storage volume 6060. The internal port 6400 can be coupled to an external fitting 6422 with the external fitting 6422 being outside of the storage volume 6060 and the internal port 6400 being disposed within the storage volume 6060. In some embodiments, the internal port 6400 and the external fitting 6422 are a contiguous component. Either one or both of the internal port 6400 and the external fitting 6422 can be connected to or sealed to the layers 6100 and 6200 at the opening 6420.

In some embodiments, the internal port 6400 allows biological material to flow into the storage volume 6060 at lower pressures than would be required in the absence of the internal port 6400. For example, traditional solutions include pressurizing the flow of biological material into container enough to separate layers of material that naturally stick together. By introducing the biological material via an internal port 6400, the layers are already partially separated due to the internal port 6400, and creating the remaining separation is accomplished at reduced pressures compared to a system without the internal port 6400.

In some embodiments, the internal port 6400 includes a tube having a wall 6406 that defines a hollow interior passage (not shown) suitable for passage of the biological material G. In accordance with some embodiments, the internal port 6400 includes one or more apertures (e.g., 6402, 6404) which open into the storage volume 6060. In one example, the only aperture is an end opening 6402. In another example, a set of apertures 6404 extends through the wall along a portion of the length of the internal port 6400. Having multiple apertures allows for lower resistance for incoming flow of biological material. Having multiple apertures allows for easier egress out of the biological material back out of the storage volume 6060. A single hole at the end of the internal port 6400 would make it more difficult to direct the biological material to the single aperture and into the internal port 6400 for egress out of the storage volume 6060.

In some embodiments, the apertures 6404 can be through-holes extending through the wall 6406. In one example of such an embodiment, the through-holes can extend through the wall 6406, across the passageway, and out the wall 6406 again. In this manner, there are two opposing holes at each longitudinal position along the tube. In other examples, the apertures could be single holes positioned along the length of the internal port 6400.

In some embodiments, the internal port 6400 includes a structure that is more rigid than the layers 6100 and/or 6200. As indicated above, the container 6000 is a flexible container with the layers 6100 and 6200 able to collapse together and even stick together. The internal port 6400 includes a stable structure suitable to hold the layers apart, at least in the proximity of the internal structure. In some embodiments, the internal port 6400 is of a different material than that of the layers 6100 and 6200. In some embodiments, the internal port 6400 is of the same material as the layers 6100 and 6200 but includes a different thickness or structure such that it maintains a cross-sectional shape.

In some embodiments, the internal port 6400 has a length that is less than the longitudinal length of the storage volume 6060. In some embodiments, the length of the internal port 6400 relative to the storage volume 6060 can vary. It is shown as being a ratio of about 0.75, but could be between about 0.25 and about 0.95, etc. Just cover some amount of range. In some embodiments, the length of the internal port 6400 is from about 25% to about 95% of the longitudinal length of the storage volume 6060. In one embodiment, the length of the internal port 6400 is about 75% of the longitudinal length of the storage volume 6060.

As discussed above, the flexible container 6000 includes at least a first opening 6420 suitable for one or more of entry and egress of a biological material into or out of the flexible container. In some embodiments, the flexible container 6000 includes an external port 6500. The external port 6500 can define an opening 6520 through the seal 6300 on the first end portion 6010 of the flexible container 6000. The external port 6500 can include an external fitting 6522.

In accordance with some embodiments, the 6422 can be connected to a y-connector 6700 suitable to connect to separate material sources. One material source can be the biological material F1. The other material source can be a treatment material T1. In one example, as shown, the treatment material T1 can be a gas source such as a CO2 exchange. The y-connector can include a flow control 6710 (e.g., a valve, pinch clamp, or the like) suitable to optionally connected or disconnected the treatment material source T1 from the container 6000. Similarly, the biological material flow F1 can be optionally connected or disconnected from the container 6000 (the flow control for this is not shown). In embodiments in which the treatment material source is a gas exchange T1, the system can include an aeration filter 6720 (e.g., a 0.4-1 um filter). The aeration filter 6720 can introduce gas into the internal port 6400 allowing gas exchange (as indicated by arrows EX) with the biological material F1 already there. In some embodiments, the treatment material source T1 is connected to the same internal port 6400 as the biological material is introduced. In other embodiments, the treatment material source T1 is connected to different internal port than the biological material (see for example, container 2000 and 3000, which include multiple internal ports, with each port being useable for a separate function).

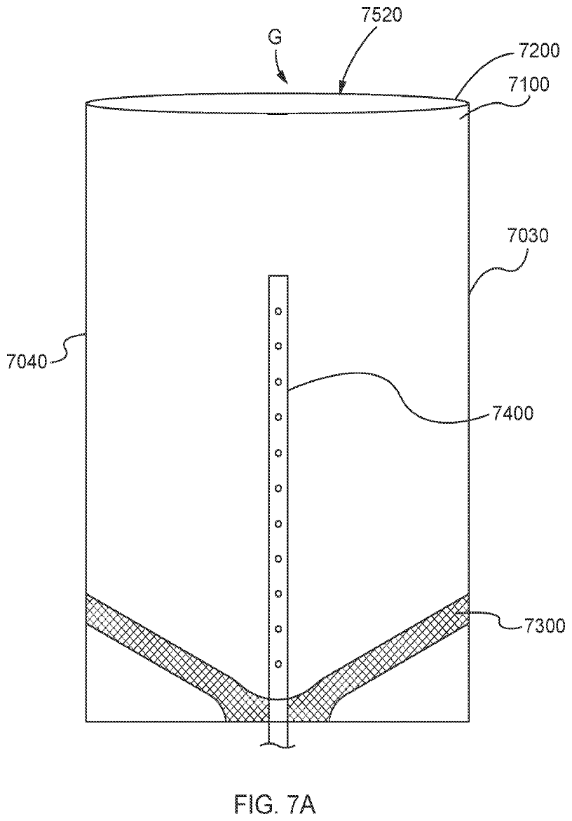

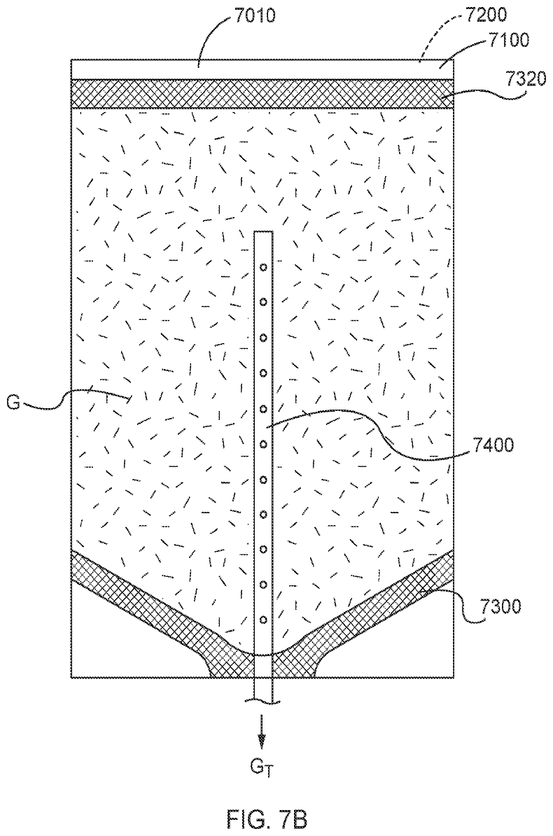

In accordance with some embodiments, a flexible container 7000 can include an internal port 7400 as shown in FIGS. 7A and 7B. The flexible container 5000 includes the various embodiments and examples discussed above with respect to flexible containers 1000, 2000, 3000 4000, 5000, and 6000. The flexible container 7000 deviates from the disclosure above with respect to the external port (e.g., 1500, 2500, 3500a, 3500b, 4500, 5500, and 6500). Here the container 7000 includes a sealable opening 7520 between the layers 7100 and 7200. The sealable opening 7520 includes an unsealed portion of the layers 7100 and 7200. The sealable opening 7520 can be substantially the width of the container 7000. In other words, the sealable opening 7520 can extend between a first side 7030 of the flexible container 7000 and a second side 7040 of the flexible container 7000. This opening width allows for larger biological material G (e.g., tissue such as bone grafts, heart valves, or other material for transplanting) to be inserted into the container 7000. Although other material such as cell cultures can still be placed in the container 7000. The biological material G is inserted through the sealable opening 7520. After filling the container (either partially or fully) with the biological material G, a seal 7320 can be applied across the container on the end 7010 thereby sealing the biological material G therein between seal 7300 and 7320. The biological material G can then be processed via the internal port 7400. For example, in some embodiments processing solutions can be flowed into or out of the container via the internal port 7400. In other embodiments, the internal port 7400 can have two ends that extend from the container (similar to the ports shown in FIGS. 4 and 5). In such embodiments, a heat exchange fluid can be circulated through the internal port (which can be sleeved to ensure fluidic isolation from the storage volume of the container) to facilitate heating (thawing) or cooling (freezing) of the biologic material G.

In some embodiments applying the seal across the sealable opening 7520 includes coupling a portion of the first layer 7100 to a portion of the second layer 7200 to hermitically seal the storage volume following the introduction of the biological material G into the storage volume. The seal can be configured as a peelable seal. The peelable seal can be configured such that the first layer 7100 can be peeled away from the second layer 7200 to expose the storage volume.

While some embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

Although some embodiments have been described as having particular features and/or combinations of components, other embodiments are possible having a combination of any features and/or components from any of embodiments as discussed above. Aspects have been described in the general context of medical devices, and more specifically tissue packaging devices, but inventive aspects are not necessarily limited to use in medical devices and tissue packaging.

1. A flexible container for storing a biological material, comprising:
a first layer coupled to a second layer via a plurality of seals to define a storage volume, the plurality of seals defines an opening between the storage volume and an external environment; and
an internal port positioned within the storage volume and in fluid communication with the opening, the internal port positioned to maintain a separation volume between the first layer and the second layer, the internal port having a wall that defines an interior passage, the interior passage being in fluid communication with the storage volume.

2-6. (canceled)

7. The flexible container of claim 1, wherein the wall of the internal port defines an aperture between the interior passage and the storage volume.

8. The flexible container of claim 7, wherein:
the internal port is fluidly coupled to a connector unit; and
the connector unit is configured to be fluidly coupled to a gas source and to a biological material source.

9. The flexible container of claim 8, wherein:
the connector unit includes a flow control member;
in a first state, the flow control member is configured to place the storage volume in fluid communication with the biological material source to facilitate delivery of the biological material from the biological material source and into the storage volume; and
in a second state, the flow control member is configured to place the storage volume in fluid communication with the gas source to facilitate the gas transfer.

10. (canceled)

11. The flexible container of claim 1, further comprising:
a sleeve positionable within the interior passage, the sleeve being configured to transition the internal port from a first transmission state to a second transmission state, wherein:
in the first transmission state, the internal port is configured to deliver the biological material into the storage volume, and
in the second transmission state, the internal port is configured to convey a treatment media.

12. (canceled)

13. The flexible container of claim 1, wherein the internal port is a first internal port and the opening is a first opening, the flexible container further comprising:
a second internal port positioned within the storage volume and in fluid communication with a second opening defined by the plurality of seals, the second internal port having a wall that defines an interior passage that is in fluid communication with the storage volume.

14-16. (canceled)

17. The flexible container of claim 1, wherein the internal port extends from the opening into the storage volume.

18. The flexible container of claim 1, wherein the internal port extends through a seal of the plurality of seals defining the opening.

19. The flexible container of claim 1, further comprising:
an external fitting coupled to the internal port, the external fitting being outside the storage volume.

20-23. (canceled)

24. The flexible container of claim 1, wherein:
at least one of the first layer or the second layer is gas permeable; and
the gas permeability facilitating a gas transfer between the storage volume and an external environment.

25. The flexible container of claim 1, wherein:
the internal port facilitates at least one of a delivery of the biological material into the storage volume or a removal of the biological material from the storage volume;
the interior passage has a cross-sectional area sized to facilitate passage of the biological material;
the wall of the internal port defines an aperture between the interior passage and the storage volume; and
the aperture is sized to facilitate passage of the biological material therethrough.

26-36. (canceled)

37. The flexible container of claim 1, wherein:
the internal port has a first cross-sectional shape on a condition that the storage volume does not contain the biological material;
the internal port is configured to have a second cross-sectional shape on a condition that the storage volume contains the biological material; and
the first cross-sectional shape is different from the second cross-sectional shape.

38. (canceled)

39. The flexible container of claim 1, wherein the opening is a first opening in fluid communication with the internal port, the flexible container further comprising:
an external port, the external port defining a second opening in the plurality of seals.

40. The flexible container of claim 39, wherein:
the external port and the second opening are positioned at a first end portion of the flexible container; and
the first opening is positioned at a second end portion of the flexible container.

41-43. (canceled)

44. The flexible container of claim 1, wherein the opening in fluid communication with the internal port is positioned at a second end portion of the flexible container the flexible container further comprising:
a sealable opening defined by the first layer and the second layer, wherein:
the sealable opening extends between a first side of the flexible container and a second side of the flexible container,
the sealable opening is positioned at a first end portion of the flexible container,
the sealable opening is configured to facilitate an introduction of the biological material into the storage volume and a removal of the biological material from the storage volume, and
the sealable opening is configured to form a seal following the introduction of the biological material into the storage volume.

45-54. (canceled)

55. A flexible container for storing a biological material, comprising:
a first layer coupled to a second layer via a plurality of seals to define a storage volume, the plurality of seals defines a first opening and a second opening between the storage volume and an external environment; and
an internal port positioned within the storage volume, the internal port having a first end portion in fluid communication with the first opening and a second end portion in fluid communication with the second opening, the internal port positioned to maintain a separation volume between the first layer and the second layer, the internal port having a wall that defines an interior passage in fluid communication with the first opening and the second opening.

56. (canceled)

57. The flexible container of claim 55, wherein the interior passage is in fluid communication with the storage volume via a gas-permeable portion of the wall of the internal port.

58-59. (canceled)

60. The flexible container of claim 55, wherein:
the interior passage is configured to receive a treatment media; and
the internal port is positioned to convey the treatment media through the storage volume to affect the biological material in the storage volume.

61. The flexible container of claim 60, wherein:
the interior passage of the internal port is fluidically isolated from the storage volume;
the treatment media is a heat transfer media; and
the heat transfer media is configured to affect a temperature within the storage volume.

62-63. (canceled)

64. The flexible container of claim 55, further comprising:
an external port, the external port defining a third opening in the plurality of seals, the external port being configured to facilitate an introduction of the biological material into the storage volume and a removal of the biological material from the storage volume.

65-68. (canceled)
